(19) United States
(12) Patent Application Publication
(10) Pub. No.: US 2005/0283193 A1
(43) Pub. Date: Dec. 22, 2005

(54) Introducer guide

(75) Inventors: Tobias Tullberg, Uppsala (SE); Dan Akerfeldt, Uppsala (SE)

Correspondence Address: FOLEY AND LARDNER LLP;SUITE 500, 3000 K STREET NW, WASHINGTON, DC, 20007, US

(73) Assignee: RADI MEDICAL SYSTEMS AB

(21) Appl. No.: 10/870,545

(22) Filed: Jun. 18, 2004

(51) Int. Cl. A61B 17/04

(52) U.S. Cl. 606/232

(57) ABSTRACT

The invention provides a medical introducer (21; 31; 41; 51; 61; 71; 81; 91; 101) having a tubular distal portion (22; 32; 42; 52; 62; 72; 82; 92; 102) with a wall (23; 33; 43; 53; 63; 73; 83; 93; 103) and a distal end opening (24; 34; 44; 54; 64; 74; 84; 94; 104) and being adapted for introduction of a haemostatic material (4) into a canal (9) in a patient's body, said canal extending from the patient's skin (13) and into the body and comprising an elongated member (15; 86; 96; 106), which is attached in the canal and extends in the canal and out from the patient's skin, wherein a guide (25; 35; 45; 55; 65; 75; 85; 95; 105) has been provided at the distal portion of the introducer, said guide being adapted for sliding engagement with the elongated member.

### FIELD OF THE INVENTION

The present invention relates generally to medical introducers, which through a wound canal are used to obtain access to blood vessels, cavities, organs or other bodily tissues in order to perform a medical operation, and in particular to a medical introducer whose distal end is provided with a guide such that the introducer can be attached to a suture or similar elongated member that extends out from the wound canal and then be advanced into the wound canal.

### BACKGROUND OF THE INVENTION

An introducer is an elongated tubular member, which in the field of medical surgery is used to gain access to a particular site within a patient's body. Usually the corresponding medical operation commences with a puncture operation in which a hollow needle is introduced at a point on a patient's skin, and is then advanced through tissues beneath the skin to the position of the organ of interest. A guide wire is then introduced through the needle, whereupon the needle is removed, and an introducer is advanced over the guide wire. After removal of the guide wire, access to the organ has now been obtained through the lumen of the tubular introducer.

The procedure described above can be used to gain access to a blood vessel for performing different types of intravascular operations. When the operation in question is completed, the puncture hole in the blood vessel wall can be sealed by a closure device, which by means of an introducer is positioned at an inner surface of the vessel wall and which is held in place by a suture or filament, which extends from the closure device, through tissue overlaying the vessel, and out of the skin surface. An example of this technique is disclosed in U.S. Pat. No. 4,744,364.

In U.S. Pat. No. 5,222,974, which is the parental application for various following applications, an inner member, which is adapted to be positioned at an inner surface of a blood vessel, is held in place by a filament, and serves as an anchor for a haemostatic material that is introduced into a punctuation canal leading to the vessel wall. Also in this case an introducer, which is an integrated part of a dedicated instrument for deploying the inner member and the haemostatic material, is utilized for accomplishing the sealing operation.

Although the introducers known in the prior art serve their intended purposes, they are not adapted to certain special applications and can therefore be further developed.

### SUMMARY OF THE INVENTION

An introducer is an elongated tubular member, through the lumen of which access is obtained to a site within a human or animal body. The introducer can be positioned in an operation involving the threading of the introducer over a guide wire. The known introducers have, however, no means that facilities the positioning of the introducer in a wound or punctuation canal out of which an elongated flexible member, such as a filament or suture, extends. This type of positioning of an introducer can, for example, be needed if an inner closure device—like the closure device disclosed in the above-mentioned U.S. Pat. No. 4,744,364—does not seal a wound or hole in a vessel wall properly, and it has therefore been concluded that extra haemostatic material should be brought into a punctuation canal leading to the hole in the vessel wall. The known introducers are not intended for this purpose, and offer consequently no means for a safe and reliable positioning of the introducer tip in close relationship to an outer surface of the vessel wall.

An object of the present invention is to provide a guided introducer, which is adapted to be positioned in a wound canal, out of which an elongated member, such as a suture or filament, extends. According to the invention an introducer is provided, whose distal end portion is provided with a guide being adapted for sliding attachment to an elongated member, which is attached in a wound canal and which extends in the wound canal and out of a patient's skin, such that the distal end of the introducer during a subsequent introduction into the wound canal is guided by the elongated member.

In a first embodiment, the opening is simply a hole through the introducer wall at a distal portion of the introducer. A suture or filament, which can be attached to an anchor positioned at an inner surface of a vessel wall and which extends through a wound canal and out from the skin of a patient, can then be threaded through this hole. When the introducer is advanced, until the tip of the introducer is positioned close to an outer surface of the vessel wall, the suture acts as a guide that guides the introducer tip through the wound canal.

To facilitate the threading of a suture through a side hole in the wall of an introducer, the introducer can be provided with a catch loop comprising a string, which in a loop extends through the side hole and out from the distal opening of the introducer. The suture can then, outside the distal opening of the introducer, be introduced through the string loop; and when the string subsequently is retracted through the introducer lumen and out from the side hole, the suture follows and can easily and comfortably be gripped by an operator.

In another embodiment, the opening is in the form of a straight slot, which can be positioned over an elongated member, such as a suture, which extends out from a wound canal.

Further exemplifying embodiments of the present invention involve various shapes of a slot. By providing, for example, a slot that extends from the distal end of an introducer, runs a distance in the proximal direction, and then back towards the distal end (such that a hook-shaped slot is formed), the risk that a suture during the introduction of the introducer slips out from the slot is reduced. To completely eliminate the risk that the introducer during the insertion looses contact with an elongated member, the introducer can be supplemented by an extra outer sheath, which is adapted to be advanced over the introducer and over the slit, with the suture being positioned therein, to lock and hold the suture in place.

### BRIEF DESCRIPTION OF THE DRAWINGS

FIG. 1 illustrates schematically a first step in an exemplifying medical procedure involving the use of an introducer assembly according to the present invention.

FIG. 2 illustrates schematically a second step in the exemplifying medical procedure of FIG. 1.

FIG. 3 illustrates schematically the final result of the exemplifying medical procedure of FIGS. 1 and 2.

FIG. 4 illustrates an introducer having a distal portion which is provided with a guide according to a first embodiment of the present invention.

FIG. 5 illustrates an introducer having a distal portion which is provided with a guide according to a second embodiment of the present invention.

FIG. 6 illustrates an introducer having a distal portion which is provided with a guide according to a third embodiment of the present invention.

FIG. 7 illustrates an introducer having a distal portion which is provided with a guide according to a fourth embodiment of the present invention.

FIG. 8a illustrates an introducer assembly comprising an introducer, the distal portion of which is provided with a guide according to a fifth embodiment of the present invention, and an extra introducer sheath in a first position.

FIG. 8b illustrates the introducer assembly of FIG. 8a, with the extra introducer sheath in a second position.

FIG. 9a illustrates an introducer assembly, the distal portion of which is provided with a guide and a catch loop, with the distal portion being in a first position.

FIG. 9b is an enlarged view of the distal portion of the introducer assembly of FIG. 9a, with the distal portion being in a second position.

FIG. 10 illustrates the distal portion of an introducer assembly which is provided with a guide according to a sixth embodiment of the present invention.

FIG. 11 illustrates the distal portion of an introducer assembly which is provided with a guide according to a seventh embodiment of the present invention.

FIG. 12 illustrates the distal portion of an introducer assembly which is provided with a guide according to an eighth embodiment of the present invention.

### DETAILED DESCRIPTION OF PREFERRED EMBODIMENTS OF THE INVENTION

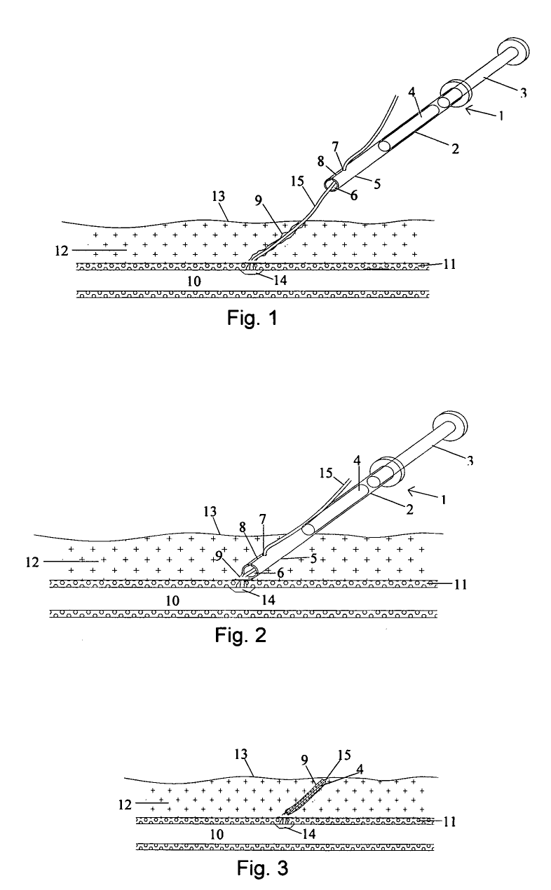

As will be appreciated from the following description, an introducer according to the present invention can be used in a variety of medical operations. For better understanding of the function and particular advantage of the invention, a special medical operation is, however, considered and described below. This medical procedure, which is illustrated in the accompanying FIGS. 1-3, serves as an illustrative example of the usefulness of an introducer having a distal end portion provided with a guide.

In the above-mentioned U.S. Pat. No. 4,744,364, a method is disclosed which involves the positioning of an inner seal at an inner surface of a vessel wall in order to seal a puncture hole therein. Although not at all discussed in the 4,744,364 patent, the inventor of the present invention has realized that to ensure proper sealing of the puncture hole it may be necessary to supplement the inner seal with a haemostatic agent or material that is introduced in a puncture or wound canal extending from the skin surface to the puncture hole in the vessel wall. Another reason for introducing a haemostatic material in the puncture canal can be to stop secondary bleeding (also referred to as oozing) occurring in the puncture canal itself. Here it should be emphasized that the first medical procedure, i.e. to position an inner seal against an inner surface of a vessel has been fully completed; and the remainder of this first medical procedure is a filament or suture (or another elongated member, such as a stem or shaft) that extends in the puncture wound and out of the skin of the patient. Besides this filament or suture (or other elongated member), the medical procedure in which an introducer according to the present invention is utilized is therefore independent of the foregoing medical procedure. This is in contrast to, for example, the medical procedure disclosed in the above-mentioned U.S. Pat. No. 5,222,974, wherein an introducer is used to position an anchor at an inner wall of a vessel, and subsequently to introduce a haemostatic material in a puncture canal leading to the vessel. In this case, the second operation, i.e. the introduction of the haemostatic material, is accomplished without removing the introducer from the puncture canal.

FIGS. 1-3 illustrate schematically how an introducer according to the present invention can be used. An introducer assembly 1, which comprises a tubular introducer 2 and a plunger 3, contains a haemostatic material 4. In this embodiment of the present invention, the introducer 2 has a distal portion 5, which ends in a distal end opening 6. According to the invention, the introducer 2 comprises further a guide in the form of a hole 7 and a slit 8. The hole 7 is provided in the wall of the introducer 2, at a rather short distance from the distal end opening 6. For example, the hole 7 may be provided within 3 centimeters of distal end opening 6, preferably within 2 centimeters, such as at about 1 centimeter. The slit 8 is also provided in the wall of the introducer 2, and extends from the distal end opening 6 to the hole 7.

FIG. 1 illustrates the first step in an exemplifying medical procedure in which the introducer assembly 1 is going to be used for introducing haemostatic material in a wound canal 9, which extends from a blood vessel 10, through a blood vessel wall 11, and through tissue 12 and skin 13 overlaying the blood vessel wall 11. An inner seal (or other inner member) 14 has, in a foregoing medical procedure, been positioned at an inner surface of the vessel wall 11 to seal the wound in the vessel wall 11, i.e. to stop bleeding from the blood vessel 10. The inner seal 14 is held in place by a suture 15 that extends from the seal 14, throughout the wound canal 9, and out from the skin 13. The slit 8 in the distal portion 5 of the introducer 2 has outside the skin 13 been threaded over the suture 15, such that the suture 15 extends from the lumen of the introducer 2 and out from the hole 7 in the wall of the introducer 2.

FIG. 2 shows the result of a second step in the exemplifying medical procedure. The second step involves the introduction of the distal portion 5 of the introducer 2 into the wound canal 9. To accomplish this second step, a doctor pushes the distal portion 5 of the introducer 2 into the opening of the wound canal 9 at the skin 13. During the advancement of the introducer assembly 1, the suture 15, which is held fairly taught, runs in the hole 7 and thereby steers the distal end opening 6 of the introducer 2 towards the vessel wall 11.

FIG. 3 illustrates the final result of a third step of the medical procedure. In this third step, the plunger 3 is pushed forward, and presses thereby the haemostatic material 4 out from the distal end opening 6 of the introducer 2. At the same time, the introducer 2 is retracted, which leaves the haemostatic material 4 in the wound canal 9. Before ending the third step, the suture 15 is cut at the surface of the skin 13.

Here it should be emphasized that the foregoing medical procedure—in which the inner seal 14 was positioned inside the blood vessel 10—is not part of the medical procedure in which an introducer according to the present invention is used. However, a prerequisite for the use of an introducer according to the present invention is that an elongated member, such as a suture or filament, extends in a canal inside the body of the patient and out of the patient's skin. The elongated member must also be attached inside the patient's body, such that the elongated member can guide the introducer within the canal. From the present invention's point of view it is not important how the canal originally was formed, i.e. whether it is an artificially created wound canal (e.g. a punctuation canal) or a natural canal (e.g. the urethra). Likewise, it is of no importance how the elongated member (e.g. a suture or filament) is attached; it can be attached to another member (like an inner or outer seal), or it can have been directly attached to tissues within the body by, for example, sewing or gluing. Further, the elongated member can have any shape as long as a guide provided at a distal portion of an introducer according to the invention can be threaded over it. It is, for example, conceivable that the elongated member is not a filament or suture, but a stem or shaft extending from an inner or outer seal positioned at a vessel wall. From the discussion above, it also follows that the invention is not restricted to an introducer. As stated above, the characteristic features of an introducer are that it is elongated and tubular; and the invention is therefore applicable to any elongated medical instrument having a hollow distal portion being adapted for introduction into a canal in a patient's body. Besides introducers, catheters, hollow dilators, drains and other types of tubular instruments are examples of such elongated and at least partially tubular medical instruments adapted to be inserted into a human or animal body.

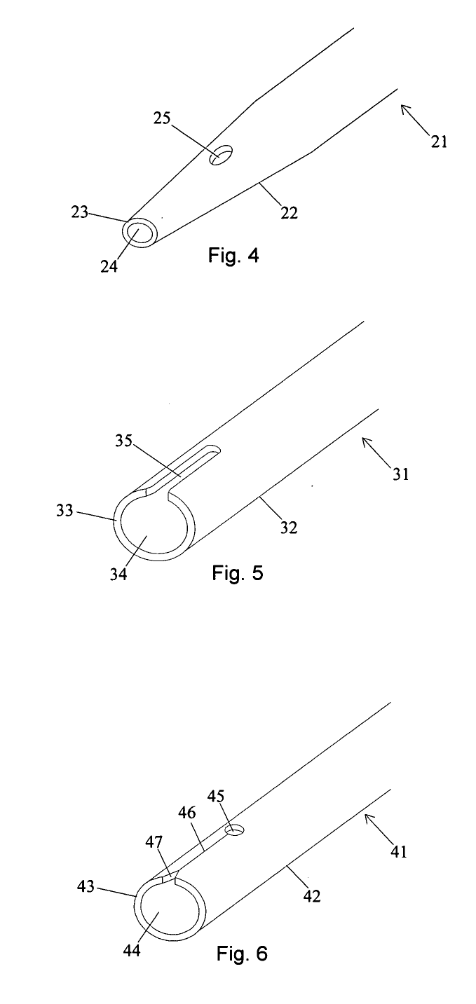

With these basic prerequisites established, a guide for an instrument being adapted for introduction into a bodily canal can—within the scope of the present invention—adopt many different shapes. From the above, it should also have been appreciated that the invention is related to the design of the distal portion of said insertion instrument. In other words: the proximal portion of an insertion instrument according to the invention can adopt any shape. The proximal portion can, for example, end in a straight-cut end, or the proximal end can be provided with a hub for attachment to another instrument. In particular, an introducer according to the invention can be a separate device, or be an integrated part of a larger assembly. Below different embodiments of the distal portion of an introducer according to the present invention will be described in conjunction with FIG. 4 to FIG. 9.

The perhaps simplest way of producing a guided introducer according to the present invention is illustrated by the first embodiment disclosed in FIG. 4. Here an introducer 21, which has a hollow distal portion 22 with a wall 23 and a distal end opening 24, comprises a guide in the form of a hole 25, which has been made through the wall 23 in the distal portion 22. As was described above in conjunction with FIGS. 1-3, an elongated member (not shown in FIG. 4), which extends in a canal in a patient's body and out from his/her skin surface, can then from the lumen of the introducer 21 be threaded through the hole 25. When the introducer 21 subsequently is introduced into this canal, the elongated member acts as a guide that guides the introducer 21 into the canal. To facilitate the introduction of the introducer 21 into the canal, the distal portion 22 of the introducer 21 has been given a shape that tapers towards the distal end opening 24. As was briefly mentioned in the background section, most introducers known in the prior art are designed to be introduced into a bodily canal in co-operation with a dilator. This is in contrast to an introducer according to the present invention, which mainly is intended to be introduced into a canal without the aid of such a dilator. It can therefore be appreciated that a tapered distal portion in many applications provides a significant advantage, although such a tapered distal portion is not crucial for the present invention. A tapered distal portion could be provided for all embodiments of the present invention described herein.

FIG. 5 discloses a second embodiment of an introducer 31 according to the present invention. The introducer 31 has a distal portion 32 with a wall 33 and a distal end opening 34. In this embodiment, a guide is provided in the form of a slot 35, which extends from the distal end opening 34 and a short distance backwards in the longitudinal direction of the introducer 31. In use, the slot 35 is positioned over an elongated member (not shown in FIG. 5) that extends in, and out from a bodily canal. When the introducer 31 is advanced into the canal, the slot 35 slides on the elongated member, which thereby acts as a guide for the introducer 31.

In FIG. 6 a third embodiment of an introducer 41 according to the present invention is illustrated. The introducer 41, which is similar to the introducer 2 described in conjunction with FIGS. 1-2, comprises a distal portion 42 having a wall 43 and a distal end opening 44. Here, a guide for the introducer 41 comprises a hole 45 and a slit 46. The hole 45 has, a short distance from the distal end opening 44, been made through the wall 43, and the slit 46 extends from the distal end opening 44 to the hole 45. The distal portion 42 is preferably somewhat elastic, such that an elongated member (not shown in FIG. 6), which extends in, and out from a bodily canal, can be squeezed into the slit 46 and moved up to the hole 45. The elongated member can thereby be positioned in the hole 45 without actually threading the elongated member through the hole 45. To facilitate the squeezing of the elongated member into the slit 46, the slit 46 ends in a small steering recess 47 being provided in the wall 43 at the distal end opening 44. Such a steering recess 47 can be in the shape of the letter V, with the apex of the letter V pointing inwards, to thereby steer an elongated member into the continuing slit 46. A similar steering recess could be provided also for other embodiments of the present invention.

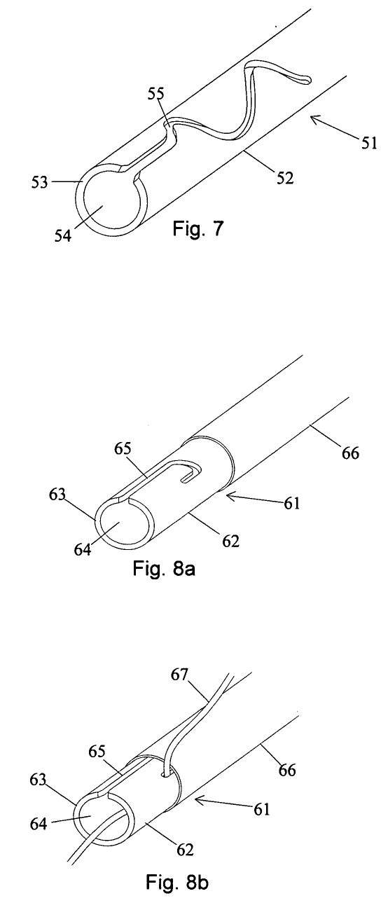

Returning now to the second embodiment shown in FIG. 5. A particular advantage of this second embodiment of the invention is that it is very easy to position the slot 35 over an elongated member. A potential disadvantage—at least in certain applications —may, however, be that the slot 35 can slip off the elongated member during the corresponding introduction procedure. To reduce the risk that an introducer slips off an elongated member during the advancement of the introducer over the elongated member, a guide in the form of a slot having a more elaborated shape can be provided. FIG. 7 illustrates an example of such a slot. Here an introducer 51, which has a distal portion 52 with a wall 53 and a distal end opening 54, is provided with a guide in the form of a slot 55 in the wall 53. The slot 55 commences at the distal end opening 54, and takes a winding or spiralling path in the proximal direction of the introducer 51. In use, the winding slot 55 is threaded and manoeuvred over an elongated member (not shown in FIG. 7), which extends in, and out from a bodily canal, until the elongated member is positioned at the proximal end of the winding slot 55. During a subsequent introduction of the introducer 51 into this canal, it is highly unlikely that the winding slot 55 slips off the elongated member.

In FIG. 8a, another example of a curved slot is presented. In this embodiment of the present invention, an introducer 61 having a distal portion 62 with a wall 63 and a distal end opening 64 is provided with a slot 65 in the wall 63. The slot 65 starts at the end opening 64, goes a distance in the proximal direction before turning back towards the distal end opening 64. In this way a slot 65 having the shape of a fishing-hook is formed. When an elongated member (not shown in FIG. 8a) has been introduced in the slot 65 and placed at its end, the risk that the slot 65 during a subsequent introduction procedure slips off the elongated member is significantly reduced. To completely eliminate this risk, an introducer according to the present invention can be provided with an extra outer sheath. As can be seen in FIG. 8a, an outer sheath 66 has been arranged over the introducer 61. The functioning of the outer sheath 66 is illustrated in FIG. 8b and described below.

FIG. 8b illustrates the introducer 61 of FIG. 8a in a state in which an elongated member 67, here in the form of a suture 67, has been introduced in the slot 65 and positioned at the end of the hook-shaped slot 65. In order to eliminate the risk that the suture 67 during an introduction procedure slides out from the slot 65, the outer sheath 66 has been moved forwards such that the outer sheath 66 covers the most proximal portion of the hook-shaped slot 65. From FIG. 8b it is thereby apparent that with the outer sheath 66 in this position the elongated member 67 cannot slip out from the slot 65 during a corresponding introduction procedure.

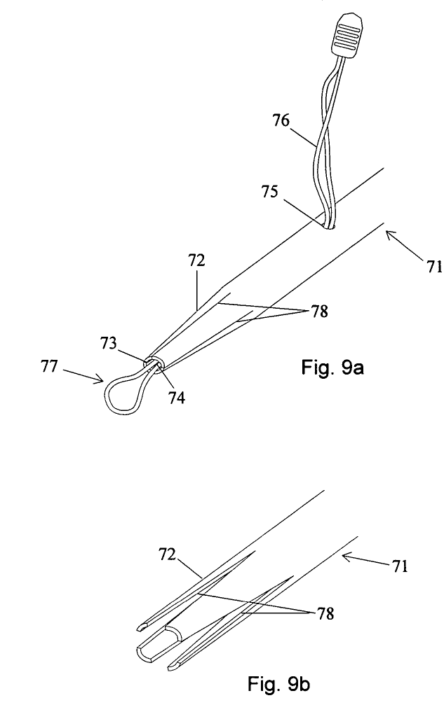

Returning now to the first embodiment shown in FIG. 4. The potential problem discussed above that an elongated member during an introduction procedure slips out from a guide provided in the distal portion of an introducer is apparently not present in the first embodiment illustrated in FIG. 4, where the guide simply comprises a hole 25 through the wall 23 of the distal portion 22 of the introducer 21. A potential disadvantage with this embodiment is, however, that it may be difficult to thread an elongated member through the hole 25. To facilitate the threading of an elongated member through a hole made in the wall of an introducer, an introducer according to the invention can be provided with a catch loop. An example of such a catch loop is illustrated in FIG. 9a. Here an introducer 71, which has a hollow tapered distal portion 72 with a wall 73 and a distal end opening 74, comprises a guide in the form of a hole 75, which has been made through the wall 73 in the distal portion 72. A double string 76 has from the outside been introduced through the hole 75 and advanced in the lumen of the introducer 71 until a loop of the double string 76 extends out from the distal end opening 74, such that a catch loop 77 is created outside the distal end opening 74. By introducing an elongated member (not shown in FIG. 9a) into the catch loop 77 and subsequently retracting the catch loop 77 out from the hole 75, with the elongated member following, a comfortable way of threading an elongated member through the hole 75 has been achieved.

As can be seen in FIG. 9a, the tapered distal portion 72 has further been provided with a number of longitudinal slits 78, which extend from the distal end opening 74 and some distance along the longitudinal length of the introducer 71. The purpose of these slits is best illustrated in FIG. 9b, which shows the distal portion 72 of the introducer 71.

FIG. 9b illustrates that by providing these longitudinal slits 78, and by making the introducer 71 from a resilient material, the tapered distal portion 72 can be dilated, something which is advantageous when, for example, haemostatic material is to be introduced into a bodily canal, and the haemostatic material is therefore pressed through the distal portion 72. Such longitudinal slits could also be provided for the other embodiments of the present invention.

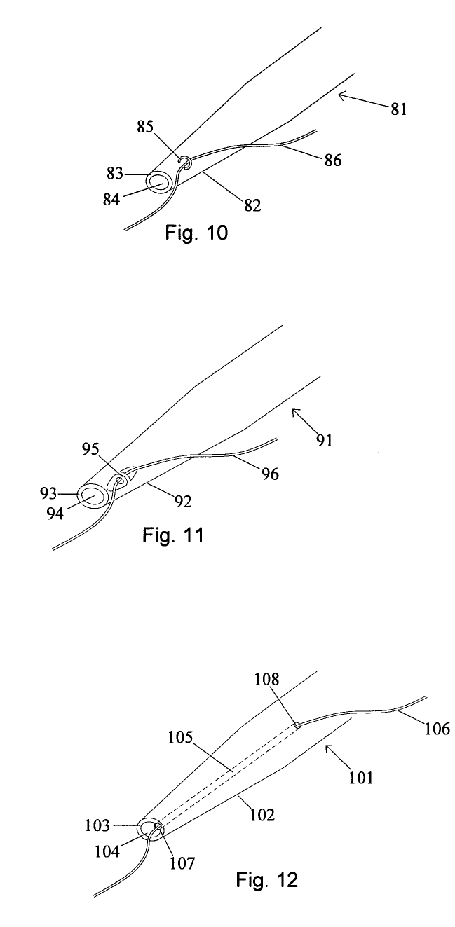

In the embodiments described above, a guide for a medical introducer has been in the form of an opening, e.g. a hole, a slit or a slot, provided in the wall of the introducer. According the present invention, a guide can also be provided on the outside of the introducer wall, or in the introducer wall itself. In FIG. 10 is illustrated an example of such a guide. Here, an introducer 81 has a distal portion 82 with a wall 83 and a distal end opening 84. In this embodiment, a guide is provided in the form of an external ring or loop 85, which is arranged on the outside surface of the wall 83. This external loop 85 is preferably disposed a short distance, e.g. a few millimetres, from the distal end opening 84. In use, an elongated member—here in the shape of a suture 86 that extends in, and out from a bodily canal—is threaded through the loop 85, and when the introducer 81 subsequently is advanced into the canal, the loop 85 slides on the elongated member, which thereby acts as a guide for the introducer 81. To not obstruct the introduction of the introducer 81 and to not damage tissue, the external ring or loop 85 is preferably made from a flexible material, such as a thread.

An external guide can also be integrated into the wall of an introducer. In FIG. 11 is shown an example of such an integrated external guide. Here, an introducer 91 has a distal portion 92 with a wall 93 and a distal end opening 94. In this embodiment, a guide is provided in the form of an external ring or loop 95, which is formed in the wall 93 of the introducer 91. Preferable the ring 95 is formed by cutting away material on both sides of a small portion and then creating a hole in this small portion. In this way, the external ring or loop 95 does not constitute a part that protrudes out from the overall introducer circumference. This external ring 95 is preferably disposed a short distance, e.g. a few millimetres, from the distal end opening 94. In use, an elongated member—here in the shape of a suture 96 that extends in, and out from a bodily canal—is threaded through the ring or loop 95, and when the introducer 91 subsequently is advanced into the canal, the ring 95 slides on the elongated member, which thereby acts as a guide for the introducer 91.

The ring or loop 95 illustrated in FIG. 11 can be regarded as a short longitudinal canal through which an elongated member can be threaded. In FIG. 12, this concept has been further developed. Here, an introducer 101 has a distal portion 102 with a wall 103 and a distal end opening 104. In this embodiment, a guide is provided in the form of a canal 105, which extends longitudinally in the introducer wall 103. The canal 103 can start close to the distal end opening 104, and in particular—as in the embodiment shown in FIG. 12—an entrance opening 107 of the canal 103 can be adjacent to the distal end opening 104 such that the distal end opening 104 and the entrance opening 107 of the canal 103 are directed in the same direction. From the entrance opening, the canal 103 runs a short distance proximally in the longitudinal direction of the introducer 101 before ending in an exit opening 108. In use, an elongated member—here in the shape of a suture 106 that extends in, and out from a bodily canal—is threaded through the canal 105, and when the introducer 101 subsequently is advanced into the canal, the canal 105 slides on the elongated member, which thereby acts as a guide for the introducer 101.

Although the present invention has been described with reference to specific embodiments, also shown in the appended drawings, it will be apparent for those skilled in the art that many variations and modifications can be done within the scope of the invention as described in the specification and defined with reference to the claims below. For example, an introducer according to the present invention is adapted for introduction of a variety of different haemostatic materials, agents, and substances. As mentioned above, the proximal end of an introducer according to the invention may be provided with a hub or connection, such as a luer-lock fitting, for connection to a syringe which can be pre-loaded with a suitable haemostatic substance, such as a hydrogel formed by chitosan, hyalyronic acid, hemi-cellulose, or similar carbohydrates, or any type of synthetic modification (e.g. cross-linking) thereof. Some these haemostatic materials and substances perform to the haemostasis by mechanically stop bleeding into an incision canal, i.e. these materials swell in contact with a body fluid such that the material occupies the available space within the incision canal and thereby prevents blood or other fluids from entering into the incision canal. Other haemostatic agents promote the clotting of the blood, while still other haemostatic agents cause vasoconstriction. Non-limiting examples of haemostatic agents that can be used in an elongated member according to the invention are: collagen, chitin and chitosan, thrombin, gelatine, oxidized regenerated cellulose, aprotinin, tranexamic acid, aminocaproic acid, desmopressin, vitamin K, factor VIIa, factor VIII, vasopressin, and conjugated oestrogen, or combinations thereof.

1. A medical introducer having a tubular distal portion with a wall and a distal end opening and being adapted for introduction of a haemostatic material into a canal in a patient's body, said canal extending from the patient's skin and into the body and comprising an elongated member which is attached in the canal and extends in the canal and out from the patient's skin, wherein a guide has been provided at the distal portion of the introducer, said guide being adapted for sliding engagement with the elongated member.

2. A medical introducer according to claim 1, wherein the guide comprises a hole through the wall of the introducer, through which hole the elongated member can be threaded.

3. A medical introducer according to claim 1, wherein the guide comprises a slot, which can be positioned over the elongated member.

4. A medical introducer according to claim 3, wherein a steering recess is provided at the distal end opening, which steers the elongated member into the slot.

5. A medical introducer according to claim 1, wherein the guide comprises a hole and a slit, into which the elongated member can be squeezed and advanced to the hole.

6. A medical introducer according to claim 5, wherein a steering recess is provided at the distal end opening, which steers the elongated member into the slit.

7. A medical introducer according to claim 1, wherein the guide comprises a slot taking a winding or spiralling path in the wall of the medical introducer.

8. A medical introducer according to claim 7, wherein a steering recess is provided at the distal end opening, which steers the elongated member into the slot.

9. A medical introducer according to claim 1, wherein the guide comprises a slot in the wall of the introducer, which slot commences at the distal end opening, runs a distance in the proximal direction before going back towards the distal end opening, such that a hook-shaped slot is formed.

10. A medical introducer according to claim 9, wherein a steering recess is provided at the distal end opening, which steers the elongated member into the slot.

11. A medical introducer according to claim 1, wherein the guide comprises an external loop, which is provided on the wall of the introducer.

12. A medical introducer according to claim 1, wherein the guide comprises an external loop, which is integrated in the wall of the introducer.

13. A medical introducer according to claim 1, wherein the guide comprises a canal, which runs a distance in the wall of the introducer.

14. A medical introducer according to claim 1, wherein the distal portion of the introducer tapers towards the distal end opening.

15. A medical introducer according to claim 1, wherein the introducer is made from an elastic or flexible material.

16. A medical introducer according to claim 1, wherein the distal portion of the introducer has been provided with longitudinal slits for facilitating dilation of the distal portion.

17. A medical introducer according to claim 1, further comprising a proximal portion, wherein a hub is provided at the proximal portion.

18. A medical introducer assembly comprising an introducer according to claim 2, wherein the introducer assembly further comprises a catch loop in the form of a double string that extends through the hole, through the tubular distal portion, and out from the distal end opening, such that a catch loop is formed outside the distal end opening.

19. A medical introducer assembly comprising an introducer according to claim 9, wherein the introducer assembly further comprises an outer sleeve, which can be positioned in a first position, where the outer sleeve does not cover the hook-shaped slot, and in a second position, where the outer sleeve covers a proximal portion of the hook-shaped slot, to thereby prevent an elongated member positioned at the end of the hook-shaped slot from sliding out from the hook-shaped slot.

20. A medical introducer having a tubular distal portion with a wall and a distal end opening and being adapted for introducing haemostatic material in a wound canal leading from a patient's skin, through tissue beneath the skin and to a puncture hole in a wall of a vessel, said canal comprising an elongated member extending from the outside of the patient's skin and through the canal and being connected to an element which is placed in contact with the wall of the vessel, wherein a guide is provided in the distal portion of the introducer, which guide outside the patient's skin can be connected to the elongated member, such that when the introducer is introduced into the canal, the elongated member guides the distal end opening towards the puncture hole in the wall of the vessel.
